(19) United States
(12) Patent Application Publication
(10) Pub. No.: US 2024/0384042 A1
(43) Pub. Date: Nov. 21, 2024

(54) Gel And Gel Beads Containing Polyvinyl Alcohol, Polyurethane And Immobilized Substances

(71) Applicant: GRAY PORTAL LLC, Flushing, NY (US)

(72) Inventors: Sz-Chwun HWANG, Hsinchu City (TW); Hsin-Yi HO, Hsinchu City (TW); Shih-Wei HUANG, Hsinchu City (TW)

(21) Appl. No.: 18/696,378

(22) PCT Filed: Jun. 3, 2020
(86) PCT No.: PCT/US2020/035822

§ 371 Date: Mar. 28, 2024

Related U.S. Application Data
(60) Provisional application No. 63/003,516, filed on Apr. 1, 2020.

(51) Int. Cl. C08J 3/075; B01J 13/00; C02F 3/10; C08K 3/30; C08K 3/32; C08K 3/38; C08L 29/04; C08L 75/04; C12N 11/04; C12N 11/084; C12N 11/093

(52) U.S. Cl. CPC C08J 3/075; B01J 13/0065; B01J 13/0069; C02F 3/108; C08K 3/30; C08K 3/32; C08K 3/38; C08L 29/04; C08L 75/04; C12N 11/04; C12N 11/084; C12N 11/093; C08J 2329/04; C08J 2475/04; C08K 2003/3045; C08K 2003/321; C08K 2003/387

(57) ABSTRACT

Polyvinyl alcohol (PVA) gel and polyurethane (PU)ZPVA gel and gel beads, methods for making gel and gel beads with immobilized substances such as microorganisms, cells, enzymes, and/or other materials, methods for using gel and gel beads in various applications (e.g., wastewater treatment), and apparatus for manufacturing such gel and gel beads, are described.

### CROSS-REFERENCE TO RELATED APPLICATION

This application claims the benefit of U.S. Provisional Patent Application No. 63/003,516, filed Apr. 1, 2020, which is incorporated by reference herein in its entirety.

### FIELD OF THE INVENTION

The invention relates to polyvinyl alcohol (PVA) containing gel and gel beads that optionally may contain polyurethane (PU), methods for making gel and gel beads, methods for immobilizing substances such as microorganisms, cells, enzymes, and/or other materials in gel and gel beads, and methods for using such gel and gel beads in applications.

### BACKGROUND OF THE INVENTION

The use of immobilized microorganisms and enzymes has been considered as an alternative technology for wastewater treatment to replace conventional suspension sludge (SS) systems, such as an activated sludge system (Dumitriu and Chornet, 1998). Entrapment of microorganisms and enzymes in polymeric gel has been done previously but with various disadvantages (Kuraray Co., Ltd., 2012; Aslam et al., 2018). For example, there have been many drawbacks to using PVA for immobilization, such as a resulting weak physical structure and significant adhesion problems. The leakage of PVA gel from the surface of beads often occurred under mechanical agitation from water flow or aeration. A significant PVA biological decomposition problem was caused by entrapped microorganisms such as denitrifiers, e.g., Pseudomonas, etc. Such disadvantages rendered the lifetime of the PVA gel beads to a limited useful period (e.g., a few months for some applications).

Some modifications of PVA gel beads involved a modification of the chemical structure of PVA gel beads using acetalization (Kuraray Co., Ltd., 2012), etherification (Schmidt et al., 1934) or other modifications (Aslam et al., 2018). Although this functional group modification might make PVA gel beads stronger to resist microbial decomposition, there are several drawbacks of these attempts, including that (1) they are not cost effective and require significant amounts of energy, (2) they may use aldehydes, especially glutaraldehyde, which are toxic to microorganisms, (3) common microorganisms cannot survive under certain process conditions that were applied, for example at pH<3 and temperatures of 40°-80° C., (4) the weak physical structure produced causes compaction in filters, and (5) the formation of gel beads still relies on alginate, etc., in a calcium ion solution. The structure of alginate is easily disintegrated into pieces because the calcium ion in the center of chelates can be withdrawn and replaced by phosphates in, for example, natural water. Also, alginate itself is easily decomposed by microorganisms as a carbon source.

Some previous reports suggested environmentally friendly modifications of the immobilization process using a particular PVA-boric acid method. Hwang suggested in U.S. provisional 62/856,328 (Jun. 3, 2019) that adding sodium chloride, calcium chloride and magnesium sulfate at the end of the immobilization process would improve the surface strength and solve the adhesion problem. However, it did not prevent the significant leakage of PVA gel from inside of gel beads or the end of the tadpole-shape gel beads, indicating that the major reason for the adhesion problem was still present and not solved. Hwang et al. described in a utility patent in 2014 (R.O.C. patent No. I425050) a development of ether-type anionic polyurethane (PU) gel beads. No PVA was described in those gel beads in the 2014 application. Earlier, Hwang et al. (https://www.slideserve.com/tiara/pu-pva) in 2005 disclosed PU/PVA immobilization cell beads. There is very little discussion about the PU/PVA immobilization cell beads and the PU gel beads, because of the very weak physical structure that resulted, which is not suitable for being used as a filter or for other purification or other processing methods.

U.S. Pat. No. 5,290,693 (1994) disclosed the hardening of PVA gel beads by adding phosphates. However, the gel beads disclosed there can still leak because using phosphate to harden the gel beads resulted in the gel beads not being hard enough.

An improved method for making improved gel and gel beads with immobilized substances (e.g., microorganisms such as bacteria and new substances not immobilized before) is thus needed. Such a method could be improved by using less harsh, more efficient and cost-effective processes and semi-continuous or continuous processes rather than discrete modes. The resulting characteristics of the gel and gel beads could also be improved by providing improved stability, improved viability of the immobilized substance, less leakage, improved hardness, and reinforcement.

### SUMMARY OF THE INVENTION

The embodiments of this invention include PVA and/or PU/PVA gel and gel beads with improved structures and properties (e.g., stability, hardness, reinforcement, less harsh environments, less leakage) containing immobilized substances. The embodiments of this invention also include novel and non-obvious methods (e.g., continuous, efficient processes, such as with extruders) and apparatus for making PVA and/or PU/PVA gel and gel beads containing one or more immobilized substances such as microorganisms (e.g., bacteria, algae, fungi, protozoa, etc.), cells, enzymes and/or other materials (e.g., other chemicals such as non-enzymes, other living organisms, soil, sludge, mixtures of purified, partially purified or unpurified materials).

Embodiments of these gel, gel beads, methods and apparatus may comprise several operations that can be performed serially or combined. These operations include forming a PVA slurry solution, and, optionally, combining PU with PVA to form a PU/PVA slurry solution. The PU is preferably an ether-type hydrophilic polyurethane and it may be heated or unheated before use or in the slurry solution. We are using the phrase “slurry solution” to describe a slurry and/or solution that may be complex with several components or phases present at once or at different times (e.g., components and phases present such as a slurry, a powder, a mixture, a solution, a precipitate, etc.).

Embodiments of these gel, gel beads, methods and apparatus may also comprise combining one or more anions (e.g., anion releasing compounds) with the PVA and/or PU/PVA slurry solution and then forming PVA gel and/or PU/PVA gel. The one or more anions (e.g., from added anion releasing compounds) that are used can preferably comprise sulfate, phosphate, and/or borate anions, and other suitable anions or anion releasing chemicals, as will be apparent to the person of ordinary skill in the art. These operations can be done serially or in their various combinations.

Embodiments of these gel, gel beads, methods and apparatus may optionally comprise combining the PVA and/or PU/PVA slurry solution with an etherification compound. The etherification compound is one that increases or enhances (e.g., catalyzes) ether formation and is preferably sulfuric acid or another acid.

Embodiments of these gel, gel beads, methods and apparatus may comprise then combining one or more substances, such as microorganisms (e.g., bacteria, algae, fungi, protozoa, etc.), cells, enzymes and/or other materials (e.g., other chemicals such as non-enzymes, other living organisms, soil, sludge, mixtures of purified, partially purified or unpurified materials), with the slurry solution, and then combining boric acid solution with the slurry solution with the one or more substances to immobilize and forming gel or gel beads. These operations can be done serially or combined together. The environment of the slurry solution may be improved for a given application by avoiding potentially harsh conditions that may apply. Thus, the pH of the slurry solution for some applications (e.g., microorganisms, cells) may preferably be less than about pH 7, more preferably above about pH 3, and most preferably be above about pH 5.5. Other applications may benefit from different pH ranges.

Preferred substances to be immobilized are microorganisms, cells, enzymes, non-enzyme chemicals, sludge, or mixtures of materials.

Embodiments of these gel, gel beads, methods and apparatus may also comprise forming a bead or other shape for the gel with, for example, a discrete-mode dropping apparatus, such as the apparatus that is known in the art or, more preferably, extrusion apparatus. The operation of forming a bead or other shape for the gel with an extrusion apparatus is preferably done as part of a semi-continuous or continuous process. The operation of forming a gel is preferably done by spreading it on a surface or support, such as a board, plate or reaction component, for use in a process or method.

Embodiments of these gel, gel beads, methods and apparatus may then comprise combining one or more hardening agents with the gel or gel beads containing the one or more immobilized substances. The one or more hardening agents preferably comprises a cation or a cation releasing compound, such as an alkali metal, an alkaline earth metal, other metal ion, and/or a mixture thereof. The alkali metal preferably is Li⁺, Na⁺, K³⁰ , and/or mixtures thereof. The alkaline metal is preferably Ca²⁺, Mg²⁺, and/or mixtures thereof. The other metal ion that can be used is preferably Al⁺³, Fe²⁺, Fe⁺³, Zn²⁺ and Cu²⁺, and/or mixtures thereof.

Embodiments of these gel, gel beads, methods and apparatus may then comprise, optionally, combining one or more reinforcement agents with the gel or gel beads containing the one or more immobilized substances. The optional one or more reinforcement agents preferably comprise fibers. Preferable fibers are synthetic fibers such as fibers of polyacrylic acid, polyvinyl acetate, polyacrylamide fibers and natural fibers such fibers from algae, cellulose, pulp, cotton, linen and other natural sources, and/or mixtures thereof. These operations set forth above can be done serially or in their various combinations.

Preferred embodiments of the gel beads of this invention are gel beads comprised of PVA gel and/or PU/PVA gel, including linked PVA units and/or linked PU/PVA units; gel beads with one or more immobilized substances such as microorganisms (e.g., bacteria, algae), cells, enzymes and/or other materials; gel beads of a preferred size from about 2 mm to about 6 mm, and more preferred from about 3 mm to about 5 mm, and most preferred of about 4 mm; and gel beads with less than about 10% PVA or immobilized substance leakage from the gel beads after one week of use in an application such as an aqueous solution treatment process, and more preferred with less than about 1% PVA or immobilized substance leakage from the gel beads after one week of use in an application such as an aqueous solution treatment process, and most preferred with less than about 0.1% PVA or immobilized substance leakage from the gel beads after one week of use in an application such as an aqueous solution treatment process.

In certain preferred embodiments, including those in the paragraph above, gel beads have a hardness greater than or equal to about 0.03 kg/cm², and more preferably, gel beads have a hardness greater than or equal to about 0.1 kg/cm², and most preferably, gel beads have a hardness greater than or equal to about 0.5 kg/cm². A hardness greater than or equal to about 0.03 kg/cm² may improve processability in some embodiments of this invention.

In certain preferred embodiments, including those in the two paragraphs above, gel beads pass a stress test by showing preferably less than about 5% to about 10% leakage or loss of PVA or immobilized substance, more preferably less than about 1% leakage or loss of PVA or immobilized substance, and most preferably no measurable leakage or loss of PVA or immobilized substance. One such stress test uses a velocity gradient (G≥300 s⁻¹) produced by strong agitation from coarse air bubbles for one week. Additional stress tests can be applied that mimic or relate to the given application in which the gel beads will be used to measure the leakage or loss of PVA from the beads for a relevant period of time. The leakage and loss of PVA from gel beads can be measured by, for example, measuring PVA in the solution, observation of a solution of the gel beads, observation of foaming, or measuring COD (chemical oxygen demand), for example, in a solution of reverse osmosis purified water.

Applications for the embodiments of this invention include using the PVA and/or PU/PVA gel or gel beads in various substrate and aqueous solution treatment, purification processes and manufacturing processes, as examples. These applications can comprise applying or otherwise combining the gel or gel beads of this invention that contain immobilized substances such as microorganisms (e.g., bacteria, algae, fungi, protozoa, etc.), cells, enzymes and/or other materials (e.g., other chemicals such as non-enzymes, other living organisms, soil, mixtures of purified, partially purified or unpurified materials) with substrates and aqueous solutions to, among other things, for example, reduce the COD (chemical oxygen demand), reduce volatile organic chemicals, reduce the odor, denitrify, nitrify, and/or purify the aqueous solution or produce products. The person of ordinary skill in the art understands how this application and combining of the gel and gel beads of this invention to these substrates and aqueous solutions can be done in, for example, in situ or in reaction tanks, reaction plates, reaction columns, other reaction vehicles (e.g., containers, tubes), and processes and/or be incorporated into pre-existing manufacturing and purification type processes and apparatus. Thus, this method may comprise applying the gel or gel beads containing the immobilized substances to a substrate, gas or aqueous solution, treating the substrate, gas or aqueous solution with the gel or gel beads, and recovering (e.g., retrieving, reclaiming, reusing, separating, filtering, removing, bypassing, etc.) the gel or gel beads from the treated substrate or aqueous solution. These applications can apply to many different types of substrates and aqueous solution treatments, including wastewater treatment, aquaculture water treatment, aquarium water treatment, chemical process solution treatment or production, manufacturing process solution treatment or production, biofuel and biodiesel production, antibiotic process solution treatment or production, and/or other pharmaceutical process solution treatment or production. Other applications of immobilized substances, including immobilized bacteria and algae, can be used with embodiments of this invention that are known, or will be known, to a person of ordinary skill in the art (e.g., components of devices, biosensors, bioreactors, environmental mitigation and remediation (e.g., metals, gases, toxins) applications).

Advantages of the embodiments of this invention are described and apparent throughout this specification. For example, certain embodiments permit the use of semi-continuous and/or continuous processing using an extruder, which has not been applied to relevant aqueous solutions or such gel beads in particular. The disclosed pre- and post-treatment modifications of PVA-boric acid immobilization processes for substances can provide more advantageous solutions, more stability, better strength, improved adhesion properties, less leakage, better physical and chemical structure, be more environmentally friendly, be more economical, etc., compared to previous applications of gel and gel beads, such as applications concerning wastewater treatment, and new applications that were not done before because of the drawbacks of the gel and gel beads used.

### BRIEF DESCRIPTION OF THE DRAWINGS

FIG. 1 is flowchart of an embodiment of a method or process of the invention.

FIG. 2A is a schematic block diagram of a method or process for manufacturing gel beads containing immobilized substances (e.g., microorganisms, enzyme) according to an embodiment of the invention.

FIG. 2B is a schematic diagram of a conveying mechanism for a method or process for manufacturing gel beads containing immobilized substances (e.g., microorganisms, enzyme) according to an embodiment of the invention.

FIG. 2C is a right side, front view of FIG. 2B showing the porous cover combined with the cutting pieces of this embodiment.

FIG. 3 shows that the diameters of preferred embodiments of PVA gel beads decreased and the hardness increased with increased conductivity of the sodium chloride solution in the lower concentration.

FIG. 4 shows that COD (chemical oxygen demand) concentration in wastewater was reduced to below 250 mg/liter as required by a factory during a 60-day operation and the removal efficiency was about 50% using an embodiment of the gel beads of this invention.

FIG. 5 shows the COD of two systems, i.e., a suspension system (SS) and an immobilized system (IS) embodiment of this invention, at different influent flowrates.

### DETAILED DESCRIPTION OF THE INVENTION

The invention provides a method of production of immobilized substances such as microorganisms (e.g., bacteria, algae, fungi, protozoa, etc.), cells, enzymes and/or other materials (e.g., other chemicals such as non-enzymes, other living organisms, soil, sludge, mixtures of purified, partially purified or unpurified materials) in gel and gel beads using PVA and/or PU/PVA for application in many different processes involving substrates and/or aqueous solutions, such as aquarium, aquaculture, water and wastewater treatment, and in manufacturing processes and production (e.g., the biochemical, chemical and pharmaceutical industry). In particular, it can increase the surface strength of gel and gel beads and reinforce the interior structure of the gel and gel beads through pre- and post-treatments combined with PVA-boric acid cell immobilization processes. At the same time, the invention can solve the significant adhesion problems of gel and gel beads. Thus, preferred embodiments of the gel and gel beads can maintain their dispersion during processes, conveyance and/or filtering through unit operations in manufacturing factories and treatment applications and their usage in different fields.

The invention also describes methods for improved throughput and/or mass production of immobilized substances in gel and gel beads. In particular, the use of high-productivity extruders, which have not been used with aqueous solutions at room temperature (Prüße et al., 2002), is now applied to the invention's production of immobilized substances in gel beads with the advantage of continuous discharge of PVA slurry solution in the method. Otherwise, other techniques, such as a dropping technique of manufacture using dropping apparatus, can only operate in a discrete mode.

Examples of methods of producing immobilized substances of this invention, comprises:
Pretreatments: providing a PVA (powder) slurry solution, adding anions or a chemical compound which can release anions into the PVA slurry solution and/or adding a chemical compound which can develop etherification on gel and gel beads (“etherification compound”), such as sulfuric acid or other acid. An ether-type hydrophilic polyurethane can also be added in certain preferred embodiments, which may be heated or unheated. The PVA chemical-mixed slurry solution can be heated or unheated also. A gel is then formed, such as a highly viscous gel. Alternatively, or in addition, the etherification compound can be added after the heating of the PVA chemical-mixed slurry solution or before or after adding anions or a chemical compound which can release anions into the PVA slurry solution.
PVA-boric acid treatment: A gel is then formed, such as a highly viscous gel. Microorganisms or enzymes or other substances can be added to the PVA chemical-mixed slurry solution. Thus, the invention includes embodiments that provide a forming solution of boric acid and feeding the substance-mixed (e,g., microorganisms-mixed or enzyme-mixed) into the forming solution to form a plurality of immobilized substances in gel.
Optional bead or other shape formation: if gel beads are desired they can be formed by using discrete-mode dropping methods and apparatus. More preferably, because of the advantageous characteristics of the gel provided by embodiments of this invention, the gel beads or other shapes can be formed with high-throughput, semi-continuous and continuous and efficient extrusion apparatus. Gel are preferably prepared by spreading on a supported surface, such as a board or plate for use in a process or method.
Post-treatment: providing a hardening agent and placing the gel or gel beads in a solution of a cation or cation releasing compound such as an alkali group metal or alkaline earth group metal salt. The gel or gel beads can be reinforced, hardened and dispersed in further working medium. Such processes may be preferably carried out relatively easily at low cost without adding expensive natural polysaccharides, such as sodium alginate, or subjecting the immobilized substances to toxic and denaturing conditions, such as those from acids, aldehydes, and polyvalent anions.

Embodiments of this invention can also use etherification as an alternative, for example, using ether-modified PVA as a matrix with/without other interior reinforcement materials mixed together to entrap substances such as microorganisms or enzymes into PVA gel ether-beads. A PVA of about 1% to about 20% can be modified by adding from about 0.01 to about 1% w/v sulfuric acid or another acid, an etherification compound. Also, PU can be added (e.g., from about 0.1 to about 20% PU) before or after PVA dissolution at about 90° to about 120° C. These embodiments of this invention can use significantly smaller amounts of PU (less than 5%) than what was previously used. It is surprising that such small amounts of PU can stabilize and prevent the leakage of PVA gel from gel beads. The alternative of using sulfuric acid or another acid for etherification of PVA can also prevent the leakage of PVA gel from the beads.

Thus, this invention provides an optional etherification modification that can prevent the leakage of PVA gel in PVA gel beads. Mechanical strength can further be improved by adding reinforcement materials. Some reinforcement materials such as synthetic fibers (e.g., PVAc (polyvinyl acetate), PAA (polyacrylic acid), and PAM (polyacrylamide), etc.) and/or mixtures thereof, and natural fibers (e.g., algae, cellulose, pulp, cotton, and linen, etc., and/or mixtures thereof) can be added alone or in combination to further increase mechanical strength.

The esterification of this invention can be modified by adding anions to PVA or PU/PVA gel to increase the mechanical strength of PVA or PU/PVA immobilized-substance gel beads. The anions include phosphates, sulfates and boric acid/borates at concentrations between about 0.01% to about 5%.

In pretreatment, the heating time of the PVA slurry solution is preferably about 30 to about 90 minutes (more preferably about 60 minutes). Thus, for example, with this pretreatment, preferably no leakage of PVA oligomers occurs from the gel or gel beads while the gel or gel beads undergo stress testing at the end of the process using coarse air bubble aeration or a similar technique of stress testing for about a week or more. A preferred stress test used was performed by preparing a 1-liter clean air sparger; adding 100 ml of the gel beads to the sparger; filling the air sparger with reverse osmosis (RO) water to 1 liter; agitating the air sparger; setting the airflow at 1000 ml/min (so the velocity gradient G can be about or greater than 300 sec⁻¹); observing the accumulated bubble height and recording each day for one week. Bubble or foam height less than 5 cm is preferred. A more preferred stress test is to measure COD for leakage or loss of PVA. Useful testing and analysis of G is reported in “Coagulation and Flocculation in Water and Wastewater Treatment,” IWA Publishing, London, Seattle, Bratby J. (2006); reproduced in part at https://www.iwapublishing.com/news/coagulation-and-flocculation-water-and-wastewater-treatment.

In the post-treatment, after PVA and/or PU/PVA immobilized-substance gel and gel beads have been formed and removed from the boric acid solution, the gel and gel beads may be further hardened in a solution of alkali group or alkaline earth group metal salts that have concentrations of about 0.5 to about 25% for a period of time ranging between about 30 minutes to about 15 hours, preferably between about 1 to about 5 hours. Those hardening metals include Li⁺, Na⁺, K⁺, Ca²⁺, Mg²⁺. Other metal ions such as Al⁺³, Fe²⁺, Fe⁺³, Zn²⁺ and Cu²⁺ can also be used. A pH of about 4 to about 9 is preferred for the process.

In one embodiment of the invention, all of the aforementioned features are done to produce the gel or gel beads. In other embodiments of the invention, only one or more of the aforementioned features are done to produce the gel or gel beads. This flexibility is provided to improve the characteristics of the gel or gel beads overall for a given application, as some modifications may produce other weaknesses. In these embodiments, the intent is for the methods and steps to be applied to integrated solutions to achieve the maximum or optimum beneficial effects with the minimal weaknesses for the given applications.

Examples of the manufacturing equipment for gel and gel beads containing immobilized substances such as microorganisms and enzyme provided by the present invention includes a heating dissolution tank, a mixing tank, a conveying mechanism and a bead-forming tank. The heating dissolution tank contains PVA (or PU/PVA) particles, water and anions after heating to form a PVA (or PU/PVA) gel; the mixing tank contains PVA (or PU/PVA) gel mixed with substances such as microorganisms or enzymes. The conveying mechanism has a pipeline, an extrusion piece, a cutting piece and a porous cover. The pipeline has an outlet open and an inlet connected to the mixing tank. The extrusion piece is arranged in the pipeline and close to the outlet, the porous cover closes the outlet and has a plurality of openings, and the cutting piece is arranged outside the porous cover. The bead-forming tank connected to the outlet of the first pipeline fills the boric acid solution. The PVA (or PU/PVA) slurry solution is formed into a PVA (or PU/PVA) gel in the heating dissolution tank, and then mixed with one or more substances (such as microorganisms or enzymes) in the mixing tank, and then is conveyed to the bead-forming tank through the pipeline. The PVA (or PU/PVA) gel coming to the outlet of pipeline is continuously extruded from the opening of the porous cover, and the cutting piece is used for cutting the PVA (or PU/PVA) gel extruded from the opening into multiple pieces which then enter into the bead-forming tank filled with boric acid aqueous solution. The PVA (or PU/PVA) gel is then converted into a plurality gel beads containing immobilized substances in the boric acid aqueous solution of the bead-forming tank. If extrusion is not used, a dropping technique and apparatus can be applied to the gel to form the gel beads.

In certain preferred embodiments, for mass production using an extruder, the proposed modified PVA (or PU/PVA) gel is preferred to be pretreated to increase its viscosity to more than about 5000 CPS (preferably about 10000 CPS), which may be a minimum requirement for normal extruder operation of certain extruders.

The PVA or PU/PVA gel and gel beads containing immobilized substances can have a variety of uses and applications. For example, wastewater treatment, waste gas treatment, odor treatment, aquarium water treatment, aquaculture water treatment, manufacturing process solution treatment, chemical process solution treatment and production, substrate purification, pharmaceutical (drugs (e.g., antibiotics), supplements, ingredients) production, biofuel and biodiesel production and biochemical (e.g., enzymes, antibodies) production, among others.

The PVA or PU/PVA gel and gel beads containing immobilized substances can also improve the efficacy of current existing methods and processes. There is no leakage or reduced or minimal leakage of the PVA or PU/PVA gel and gel beads, an improvement of currently existing immobilized gel and gel beads. The no or reduced leakage of PVA or PU/PVA are from gel and gel beads of this invention that contain nanopores that can be entered and exited. For example, in an overgrowth algae environment in a pond, lake, reservoir, or river, the ammonia, nitrite and nitrate (NH₄—N, NO₂⁻—N, NO₃⁻—N) in the water can enter the gel and gel beads and be converted to nitrogen gas off the water by the bacteria within. Ammonia and nitrite can be converted to nitrate which can then be absorbed together with phosphorous by water plants or algae to denitrify the water. The algae, having lost its nitrogen and phosphorous source, will then gradually be reduced and the overgrowth problem resolved or reduced. The water will then return to more environmentally acceptable conditions. Another example is in wastewater treatment, organic compounds (COD) can be converted to CO₂, and nitrogen containing compounds and ammonia (NH₄) can be converted to nitrate (NO₃⁻) then to N₂ through two different kinds (aerobics or facultative anaerobe) of bacteria in the gel and gel beads to achieve efficiency of wastewater treatment in a processing plant or factory.

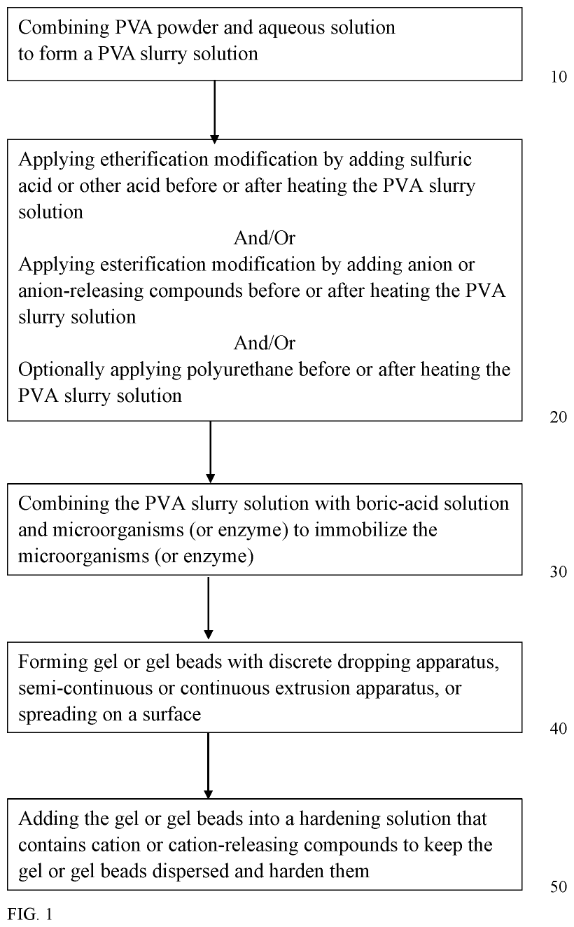

FIG. 1 relates to embodiments of novel and non-obvious pre- and post-treatment steps added to a PVA-boric acid immobilization method to make gel and gel beads of this invention. The method can be modified to perform a higher throughput and/or mass production process by replacing a discrete dropping technique with a continuously operating extruder. The different steps or operations of FIG. 1 can be applied and modified depending on the desired specification of the desired gel and gel beads to provide improved physical and chemical structure attributes and characteristics.

In pretreatment embodiments of this invention, the outer surface and/or interior structure characteristics of the gel and gel beads are characteristics that may be improved. For example, PVA-boric acid methods can poison microorganisms and/or deactivate enzymes over time. Embodiments of this invention, because of the less harsh immobilization process, can lead to expanded applications to the immobilization of different substances that could not have been done before. It is also hypothesized that the leakage of PVA using such PVA-boric acid methods is due to the weak spots on the outer surface of PVA gel beads. Thus, pre-polymerization using anions for esterification of PVA oligomer to increase the strength of interior structure is an optional and preferred embodiment. Some chemical reactions such as etherification of PVA by sulfuric acid at about 120° C. or copolymer with ether-type PU heated or unheated also can be used to seal the bead tadpole end using a dropping technique or both side cutting sections using an extruder.

During gel formation of embodiments of this invention, there are several ways to increase the viscosity of the gel (e.g., PVA gel) that may be important for successful application of higher throughput and/or mass production processes using an extruder that can be operated in a semi-continuous or continuous mode, in contrast to a discrete-mode dropping technique.

In post-treatment embodiments of this invention, cations may be added to reinforce and improve the surface characteristics of the gel and gel beads that were, for example, treated by phosphates. These cations may aid the stability of and/or otherwise improve the surface of gel and gel beads (e.g., PVA gel beads), by, for example, having increased sealing and/or increased hardness. Once the surface properties are more stable and/or otherwise improved, the adhesion problem that may apply to such gel and gel beads may be reduced or eliminated.

The subject matter of this disclosure is now described with reference to the following Examples. These Examples are provided for the purpose of illustration only, and the subject matter is not limited to these Examples, but rather encompasses all variations which are evident as a result of the teaching provided herein.

### Example 1

A PVA-boric acid method that can use a dropping technique and apparatus or extrusion technique and apparatus and that can be combined with the pretreatment 20 and post-treatment 40 50 embodiments of this invention is described herein as in FIG. 1. An aqueous solution (500 g) containing 10% by weight of PVA (99% saponification, 2400 degree of polymerization) 10 can be mixed with substances to be immobilized such as microorganisms or enzyme 30. This PVA solution can be then added into a gently stirred saturated boric acid solution drop by drop to form spherical PVA gel beads. The gel beads can be kept in the saturated boric acid solution for 60 minutes. Thereafter, the beads can be removed from the saturated boric acid solution by screener, rinsed with water, and stored in a 1-liter flask for further tests. For diameters and hardness measurements, 5 g of beads can be removed from the flask and the surface water thereon can be removed by tissue paper. Diameter and hardness can be measured for 10 beads in each example. The diameter of a bead can be measured using a digimatic caliper. Hardness is defined as the pressure added to cause a 50% change in diameter of the gel bead and measured by a force gauge (IMADA model DPX-2TR). The diameter of the beads will be between about 3 and about 5 mm in all of these embodiments.

### Example 2

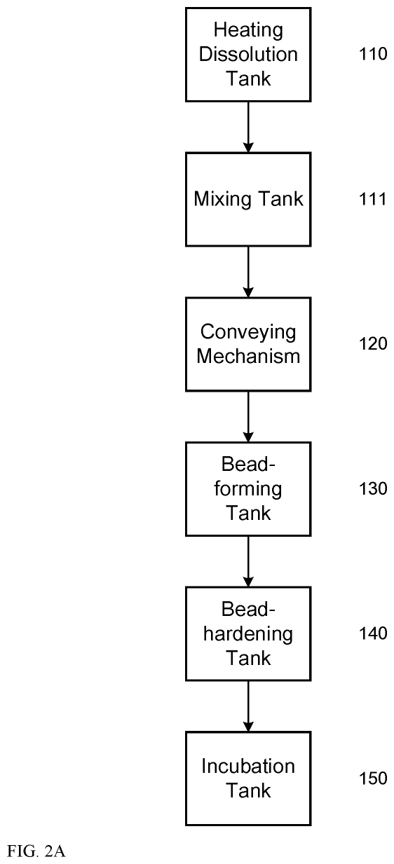

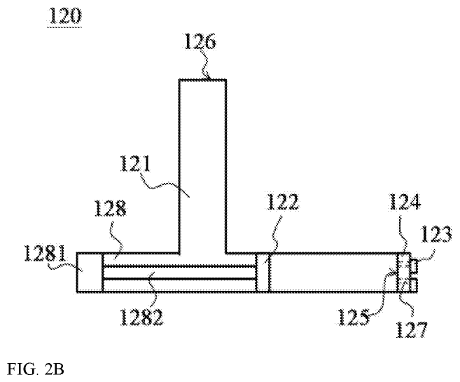

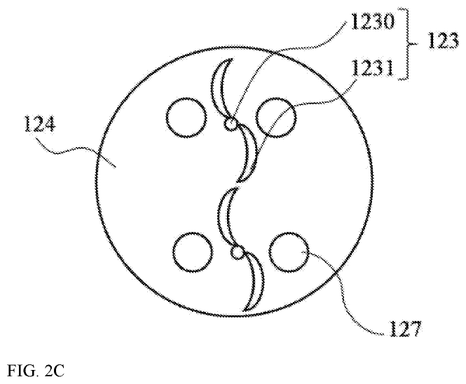

FIG. 2A is a schematic block diagram of the process for manufacturing gel beads containing immobilized substances (e.g., microorganisms or enzymes) according to an embodiment of the present invention. FIG. 2B is a schematic diagram of a conveying mechanism for an apparatus for manufacturing gel beads containing immobilized substances according to an embodiment of the present invention. FIG. 2C is a right side, front view of FIG. 2B showing the porous cover combined with the cutting pieces of the conveying mechanism. Referring to FIGS. 2A to 2C, the manufacturing apparatus 100 for immobilized substances of this embodiment includes a heating dissolution tank 110, a mixing tank 111, a conveying mechanism 120, and a bead forming tank 130. The heating dissolution tank 110 is suitable for accommodating PVA and/or PU/PVA gel and compounds that can release anions. The mixing tank 111 is suitable for containing PVA and/or PU/PVA gel with substances such as microorganisms, enzymes or other materials for immobilization.

The conveying mechanism 120 has a pipeline 121, an extrusion piece 122, a cutting piece 123, and a porous cover 124. The pipeline 121 has an outlet 125 and an inlet 126 connected to the heating dissolution tank 110 and mixing tank 111. The extrusion piece 122 is placed in the pipeline 121 and can be driven to move closer to the outlet 125. The porous cover 124 closes the outlet 125 and has a plurality of openings 127, and the cutting piece 123 is placed outside the porous cover 124.

In this embodiment, the pipeline 121 may be, for example, L-shaped, and the conveying mechanism 120 includes a buffer chamber 128 connected to the pipeline 121 and used to receive the extrusion piece 122. The buffer chamber 128 is provided with a power device 1281 and a plunger rod 1282 connected between the power device 1281 and the extrusion piece 122. The extrusion piece 122 can be a plate corresponding to the diameter of the pipeline 121. When a certain volume of PVA and/or PU/PVA gel accumulates in the pipeline 121, the extrusion piece 122 can be driven by the power device 1281 into the pipeline 121 to push the PVA and/or PU/PVA gel out of the porous cover 124 from the pipeline 121. After the squeezing operation is completed, the extrusion piece 122 can return to the buffer chamber 128. However, the present invention is not limited to this. The pipeline 121 is not limited to the L-shape, and the extrusion piece 122 may also be placed in the pipeline 121 in other ways.

In this embodiment, there may be a plurality of cutting pieces 123, and each cutting piece 123 includes a rotating shaft 1230 and a plurality of blades 1231 connected to the rotating shaft 1230. When the rotating shaft 1230 of each cutting piece 123 rotates, the blade 1230 can slide through the opening 127 of the porous cover 124 to cut the PVA and/or PU/PVA gel extruded from the opening 127 into multiple sections. The transmission shaft 1230 may be connected to a motor (not shown) and a controller (not shown) such as a microcomputer in cooperation with the action of the extrusion piece 122. However, the present invention is not limited to this, and the cutting piece 123 may also be in other forms.

The bead-forming tank 130 is connected to the outlet of the pipeline 121 in this embodiment. As can be seen from the foregoing, the bead-forming tank 130 is suitable for filling a boric acid aqueous solution. The multi-sections PVA and/or PU/PVA gel can be formed into a plurality of gel beads containing immobilized substances in the boric acid aqueous solution of the bead-forming tank 130.

In this embodiment, the manufacturing apparatus 100 for gel beads containing immobilized microorganisms or enzyme (or other substances) includes a bead-hardening tank 140 placed beside the bead-forming tank 130 and connected with the bead-forming tank 130. The bead-hardening tank 140 is adapted to receive gel beads containing immobilized substances from the bead-forming tank 130. The bead-hardening tank 140 contains the aforementioned hardening solution, and the gel beads containing the immobilized substances can be dispersed with each other and hardened in the hardening solution. Gel beads with immobilized microorganisms, enzymes or other substances need to be separated from the boric acid aqueous solution before being placed in the bead-hardening tank 140, and a sieve apparatus (not shown) is used to separate the boric acid aqueous solution from the gel beads containing immobilized microorganisms or enzymes (or other substances) and recover them. A liquid discharging device (not shown) can be arranged in the sieve apparatus for recovery of boric acid aqueous solution back to bead-forming tank 130.

In this embodiment, the apparatus 100 for immobilizing microbial gel bead particles includes a culture medium tank 150 placed beside the bead-hardening tank 140. The culture medium tank 150 is adapted to receive gel beads containing immobilized microorganisms, enzymes or other substances from the bead-hardening tank 140 and contains the culture medium. Certain of the immobilized substances that can be used, such as microorganisms immobilized in the gel beads, can be further cultivated in the culture medium tank 150. The gel beads containing immobilized microorganisms, enzymes or other substances can be stored in the culture medium tank 150 before sale.

The demonstration of an embodiment of the present invention was performed at a local machinery development factory. We first added anions (NaH₂PO₄, Na₂HPO₄, MgSO₄, and H₂SO₄, respectively) and PU to a PVA slurry solution to increase the viscosity (as seen in Table 1) to form a PU/PVA gel, and the PU/PVA gel was quickly formed into immobilized microorganisms or enzyme gel beads in the bead-forming solution containing 7% of boric acid and phosphates. Then, the immobilized microorganisms or enzyme gel beads formed in the bead-forming solution were placed into the many different bead-hardening solutions that were composed of 1% of sodium chloride, ammonium chloride, ammonium sulfate or sulfuric acid, respectively. After the immobilized microorganisms or enzyme gel beads are soaked in the bead-hardening solution for a period of time (5 hours), the beads were hardened, and the adhesion of beads did not appear.

TABLE 1

| PVA | | Anions or Other Chemicals | | | | | | | | |
|---|---|---|---|---|---|---|---|---|---|---|
| Conc | Degree of | Lot | PU | | | | | | Conc | |
| (%) | Polymerized | No | heated | NaH₂PO₄ | Na₂HPO₄ | MgSO₄ | H₃BO₃ | H₂SO₄ | (%) | Remarks |
| 10 | 2400 | 11 | 5080 | | | | | | 1 | 1. Blank is |
| 10 | 2400 | 12 | | 1565 | | | | | 1 | 1322 CPS for |
| 10 | 2400 | 13 | | | 1810 | | | | 1 | Lot No. 11-15. |
| 10 | 2400 | 14 | | | | 1886 | | | 1 | 2. All the |
| 10 | 2400 | 15 | | | | | failed | | 1 | measurements |
| 10 | 2400 | 21 | | | 1320 | | | | 2 | are performed |
| 10 | 2400 | 22 | | | 3016^{#1} | | | | 0.1 | at 50° C. |
| 13 | 2400 | 23 | | 9017 | | | | | 0.5 | 3. All the |
| 15 | 2400 | 24 | | | 175268 | | | | 0.5 | anions or |
| 10 | 2400 | 31 | | 1174 | | | | | 0.05 | chemicals |
| 15 | 2400 | 32 | | 9347 | | | | | 0.05 | were mixed |
| 20 | 2400 | 33 | | 100122 | | | | | 0.05 | with PVA and |
| 10 | 1700 | 41 | | | | | | 432 | 5 | heated at |
| 10 | 2400 | 42 | | | | | | 1105 | 0 | 90-120° C. |
| 10 | 2400 | 43 | | | | | | 2115 | 5 | |
| 10 | 2400 | 44 | | | | | | Infinite^{#2} | 1 | |

^{#1}measured after two weeks;
^{#2}when the trace amount of FeO was added.

Results in Table 1 show that the extruder can perform well when the PVA or PU/PVA gel have a viscosity of more than about 1810 CPS. Some factors affecting viscosity are PU and PVA concentration, disodium hydrogen phosphate concentration, the gel stored time, the degree of saponification, etc. Lot No. 13-14 could form small gel beads with size smaller than 1 mm that is too small for practical use in some wastewater treatment applications. One can make these small beads into fibers to be used in the pre-coated filters for treatment of aquarium water. One can use a double screw extruder for more viscous gel to make larger beads. Lot No. 23 successfully formed larger beads as shown in Table 2 which can be used directly in the wastewater treatment.

Results in Table 2 show the PVA gel containing immobilized substances in different hardening solution at a concentration of 1%. The cylinder shape of gel beads turned into spherical shapes after the gel beads were swollen in water. Adding sulfuric acid (Lot No. 52) on the surface of PVA gel beads can prevent the adhesion problem and make gel beads disperse well. It was determined that for this embodiment, chloride made bead surfaces whiter. The sulfate will make gel beads more translucent as compared to certain other hardening solutions. The “whiter” means the surface of PVA gel beads is more condensed than “translucent” is some embodiments. All of the hardening agents that show positive improvement are embodiments of qualified hardening solutions for this example. Lot 51 gel beads have a hardness at 0.037kg/cm² and size at 4.50±0.383 mm.

TABLE 2

| Lot No. | 51 | 52 | 53 | 54 | 55 |
|---|---|---|---|---|---|
| Hardening | NaCl | H₂SO₄ | NH₄Cl | NH₄SO₄ | Water |
| Solution | | | | | |
| Appearance | Dispersed | Dispersed and | Not well dispersed | Dispersed and | Beads agglomerated |
| | and white | translucent | and very white | translucent | in water. |

### Example 3

In this example, the appearance is shown of embodiments of the PVA gel beads where the 1% sulfuric acid (3.06 g concentrated sulfuric acid, 98%) and 10% PVA (30 g) were added into reverse osmosis water to make a 300-ml new method PVA slurry solution as in Tables 3 and 4. In Table 3 using NaCl as a hardening solution, which are significantly improved from gel beads disclosed in U.S. provisional application No. 63/003,516, April 2020, which is incorporated herein by reference. It is observed there that the pretreatment using sulfuric acid will make the gel beads bigger, softer, and more translucent than usual, indicating the physical structure is weak. However, these disadvantages can be corrected by embodiments of this invention so that the color can be observed whiter if the hardening cation ion concentration increases to more than 0.5%. Results in Table 4 show a similar experiment except that the hardening solution was changed to NH₄Cl instead of NaCl. The ammonium chloride can make gel beads whiter with even only the small amount of 0.1%. Both of the hardening solutions, NaCl or NH₄Cl, can keep gel beads dispersed in water. The hardness of 0.017 kg/cm² can be achieved with an average diameter of 3.32 mm±0.028.

TABLE 3

| Lot No. | 71 | 72 | 73 | 74 | 75 |
|---|---|---|---|---|---|
| PVA/H₂SO₄ | 10%/1% | | | | |
| Forming | 7% Boric Acid & Phosphate | | | | |
| Solution | | | | | |
| Hardening | 0% NaCl | 0.1% NaCl | 0.5% NaCl | 1% NaCl | 3% NaCl |
| Solution | | | | | |
| Appearance | Mostly are white, | Mostly are white, | Almost all are | All are white, | All are white, |
| in Hardening | seriously adhesive | some are | white, seriously | seriously | most adhesive, |
| Solution | | translucent, some | adhesive | adhesive | some are |
| | | are dispersed | | | dispersed |
| Appearance in | Translucent, keep | Translucent, keep | White, keep | Whit, keep | White, keep |
| Water after | dispersed, soft | dispersed, soft | dispersed, soft | dispersed, soft | dispersed, soft |
| the Hardening | | | | | |
| Size, mm | — | 5.46 ± 0.192 | — | — | 5.29 ± 0.238 |

TABLE 4

| Lot No. | 81 | 82 | 83 | 84 | 85 |
|---|---|---|---|---|---|
| PVA/H₂SO₄ | 10%/1% | | | | |
| Forming | 7% Boric Acid & Phosphate | | | | |
| Solution | | | | | |
| Hardening | 0% NH₄Cl | 0.1% NH₄Cl | 0.5% NH₄Cl | 1% NH₄Cl | 3% NH₄Cl |
| Solution | | | | | |
| Appearance | All are white | All are white | All are white | All are white | All are white |
| in Hardening | and dispersed | and dispersed | and dispersed | and dispersed | and dispersed |
| Solution | | | | | |
| Appearance in | All are white | All are white | All are white | All are white | All are white |
| Water after | and dispersed | and dispersed | and dispersed | and dispersed | and dispersed |
| the Hardening | | | | | |

### Example 4

In this embodiment, unheated neutral ether-type PU was added to a PVA slurry solution before or after heating so that the leakage of PVA oligomer in PVA gel beads will not occur under a stress test using aeration with coarse air bubbles. Table 5 shows that using 6.7% PVA and 18% PU hardened in phosphates and NaCl can make a white and glossy surface with a little hardness of 0.005 kg/cm². Lot No. 94 shows that there was no leakage of PVA oligomer under a stress test even though the hardening procedure was eliminated. Table 6 shows the gel beads significantly shrank in bead-forming solution if PU was heated and added into a PVA slurry solution. The PVA gel mixed with heated PU became opaque, like paste, and highly viscous (a viscosity of 5080 CPS measured under similar pretreatment condition as seen in Lot No. 11). Its appearance was also manifested in the rotating speed of the peristaltic pump that was used in the dropping technique for production of gel beads. Table 6 also shows that the size of PVA gel beads with heated PU changed from 2.7 mm to a larger 4 mm. Embodiments of these gel beads made with heated PU lose the glossy surface and the gel beads become softer as compared to PVA gel beads with unheated PU. Consequently, exemplary embodiments with the PU unheated provides different and more advantageous results for some applications compared 10 to heated PU. The 12% PVA with 2.75% unheated PU also showed good characteristics for the PVA gel beads as seen in Table 7 Lot No. 104.

TABLE 5

| Lot No. | 91 | 92 | 93 | 94 |
|---|---|---|---|---|
| PU/PVA | 6.7%/18% | | | |
| Hardening Solution | NaCl | Phosphates | NaCl + Phosphates | Water |
| PU Heated | No | | | |
| Hardness (kg/cm²) | Too soft to | 0.003 | 0.005 | Totally flat |
| | be measured | | | |
| Size (mm) | 3.50 ± 0.168 | 3.79 ± 0.092 | 3.78 ± 0.141 | Too soft to |
| | | | | be measured |
| Appearance | White on surface, | White on surface, | White surface, more | No leakage of |
| | very soft and | soft and elastic. | glossy than usual, | PVA gel under |
| | little elastic. | | elastic, and the most | stress test. |
| | | | hardening result in | |
| | | | this series of | |
| | | | experiments. | |

TABLE 6

| Lot No. | 101 | 101 | 103 | 104 |
|---|---|---|---|---|
| PU/PVA | 12%/2.75% | | | |
| Hardening Solution | NaCl | NaCl | NaCl + Phosphates | NaCl + Phosphates |
| PU Heated | Yes | | | No |
| Hardness (kg/cm²) | Too small to | Too soft to | 0.020 | 0.038 |
| | be measured | be measured | | |
| Size (mm) | 2.70 ± 0.212 | 3.98 ± 0.324 | 3.44 ± 0.111 | 3.81 ± 0.103 |
| Appearance | The gel shows | The gel beads | Surface is more | The gel beads |
| | translucent like | became bigger in | glossy than the | maintain a tadpole |
| | paste with high | size after swollen | other and the most | shape in water. |
| | viscosity. The gel | in water. | hardening result in | White surface. |
| | beads in NaCl | | this series of | |
| | solution is very | | experiments using | |
| | small and hard. | | heated PU. | |

TABLE 7

| | PVA | PU | | | | | |
|---|---|---|---|---|---|---|---|
| Lot | Conc | Conc | Forming | Hardening | Hardness | Size | |
| No. | (%) | (%) | Solution | Solution | (kg/cm²) | (mm) | Appearance in Water |
| 111 | 6.7 | 18 | H₃BO₃ + | H₂O | — | — | no leakage in aeration, no |
| | | | Phosphate | | | | mechanical strength, very white |
| | | | | | | | and shine. |
| 112 | 6.7 | 18 | H₃BO₃ + | NaCl | — | — | the same as above but mechanical |
| | | | Phosphate | | | | strength improved. |
| 121 | 6.7 | 18 | H₃BO₃ | NaCl | — | 3.50 ± 0.168 | white on surface, very soft and |
| | | | | | | | little elastic. |
| 122 | 6.7 | 18 | H₃BO₃ | Phosphate | 0.003 | 3.79 ± 0.092 | white on surface, soft but elastic |
| 123 | 6.7 | 18 | H₃BO₃ | NaCl + | 0.005 | 3.78 ± 0.141 | Surface more glossy than usual, |
| | | | | Phosphate | | | white, elastic, and the most |
| | | | | | | | hardening result in this series of |
| | | | | | | | experiments. |
| 101*¹ | 12 | 2.75 | H₃BO₃ | NaCl | — | 2.70 ± 0.212*² | The gel shows translucent like |
| | | | | | | 3.98 ± 0.324 | paste with high viscosity. Very |
| | | | | | | | small in NaCl solution before |
| | | | | | | | swollen in water. Very translucent. |
| | | | | | | | Smear in solution. |
| 102 | 12 | 2.75 | H₃BO₃ | NaCl + | 0.020 | 3.44 ± 0.111 | White surface, more glossy than |
| | | | | Phosphate | | | other, elastic, and the most |
| | | | | | | | hardening result in this series of |
| | | | | | | | experiments. |
| 103 | 12 | 2.75 | H₃BO₃ | Phosphate | 0.012 | 3.41 ± 0.135 | White surface, more glossy than |
| | | | | | | | other, and less elastic. |
| 104 | 12 | 2.75 | H₃BO₃ | NaCl + | 0.038 | 3.81 ± 0.103 | The gel beads maintain the tadpole |
| | | | | Phosphate | | | shape in water. White surface. |

*¹PU is heated in Lot No. 101-103.
*²Size was determinated in NaCl solution before swollen in water;

### Example 5

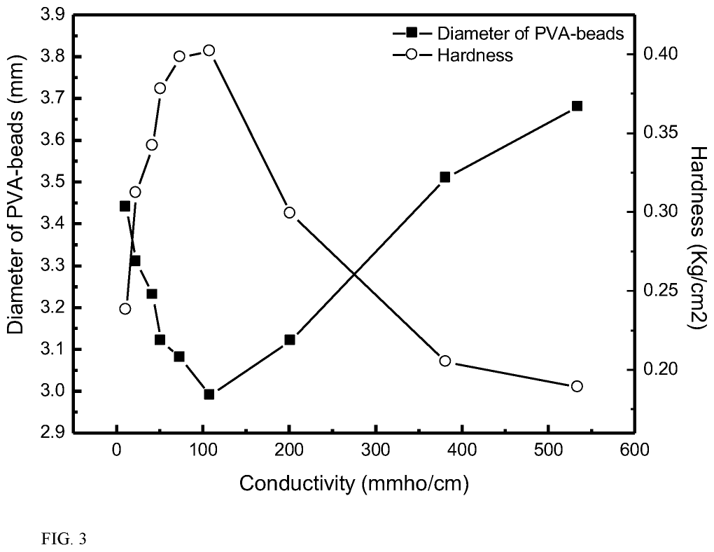

In this embodiment, the PVA gel beads pretreated with NaH₂PO₄ and processed by a PVA-boric acid method were transferred separately into aqueous solutions of NaCl with varying concentration of 0.5, 1, 2, 3, 4, 5, 10, 20 and 25% and kept therein for 60 minutes. These beads were then removed from the solutions and rinsed with water. Diameter and hardness measurements were taken for ten PVA beads from each group. The remaining beads were put in 1000-mL air sparger for aeration stress tests. During aeration, 1000 mL/min of air was aerated for a week, after which the beads were removed for physical property measurements. FIG. 3 shows that the diameters of PVA gel beads decreased and the hardness increased with increased conductivity of the sodium chloride solution in the small amount of sodium chloride. However, the opposite trend resulted when the concentration of the sodium chloride solution was higher than 5% in these embodiments. The appearances of the beads are also described in Table 8. After the aeration stress test, the beads that were hardened in solutions with concentrations between 0.5 to 25% kept a white spherical shape. The beads hardened in the solution with a concentration of 0.5% became translucent after aeration, indicating the physical structure of these particular PVA gel beads is weak. The beads which were hardened in the solution with concentrations higher than 5% adhered together after the aeration stress test. Thus, the sodium chloride with the concentration between 1 to 5% (minimum at 1%) is preferred for hardening in the use of this optional post treatment. These gel beads have some leakage of microorganisms from the gel beads.

TABLE 8

| Lot No. | 131 | 132 | 133 | 134 | 135 | 136 | 137 | 138 | 139 |
|---|---|---|---|---|---|---|---|---|---|
| Concentration | 0.5 | 1 | 2 | 3 | 4 | 5 | 10 | 20 | 25 |
| (%) | | | | | | | | | |
| Size (mm) | 3.44 ± | 3.31 ± | 3.23 ± | 3.12 ± | 3.08 ± | 2.99 ± | 3.12 ± | 3.51 ± | 3.68 ± |
| | 0.28 | 0.11 | 0.08 | 0.018 | 0.003 | 0.006 | 0.018 | 0.08 | 0.110 |
| Hardness | 0.238 ± | 0.312 ± | 0.342 ± | 0.378 ± | 0.398 ± | 0.402 ± | 0.299 ± | 0.205 ± | 0.198 ± |
| (kg/cm²) | 0.043 | 0.003 | 0.018 | 0.022 | 0.015 | 0.002 | 0.057 | 0.103 | 0.097 |
| Appearance* | Translucent | white | white | white | white | white | agglomerate, | agglomerate, | agglomerate, |
| | | | | | | | adhesive, | adhesive, | adhesive, |
| | | | | | | | white | white | white |

*Color of beads after stress test with coarse air bubble aeration

The properties of the gel beads of this example would be improved by application of the use of PU, etherification compounds and/or anions or anion releasing compounds to the making of the gel beads.

### Example 6

In this embodiment, PVA gel beads pretreated with NaH₂PO₄ and processed by a PVA-boric acid were transferred separately into aqueous hardening solutions of KCl with varying concentration of 0.5, 1, 2, and 3% and kept therein for 60 minutes. Table 9 shows that the diameters of the beads decreased with increased conductivity of the KCl solution, and the hardness of the beads also increased. After an aeration stress test, the beads which were hardened in solutions with a concentration of at least 1% maintained their white spherical appearance. However, the beads which were hardened in the solution with a concentration of 0.5% became translucent after aeration a stress test. These gel beads have some leakage of microorganisms from the gel beads.

TABLE 9

| Lot No. | 141 | 142 | 143 | 144 |
|---|---|---|---|---|
| KCl Conc. (%) | 0.5 | 1 | 2 | 3 |
| Size (mm) | 3.68 ± 0140 | 3.43 ± 0.006 | 3.27 ± 0.001 | 3.19 ± 0.002 |
| Hardness (kg/cm²) | 0.107 ± 0.027 | 0.211 ± 0.022 | 0.249 ± 0.009 | 0.299 ± 0.004 |
| Appearance* | Little | White | White | white |
| | translucent | | | |

*Color of beads after stress test with coarse air bubble aeration

The properties of the gel beads of this example would be improved by application of the use of PU, etherification compounds and/or anions or anion releasing compounds to the making of the gel beads.

### Example 7

In this embodiment, the PVA gel beads pretreated with NaH₂PO₄ and processed by a PVA-boric acid method were transferred separately into aqueous solutions of CaCl₂ with varying concentration of 0.25, 0.5, 1, 2, 3, 5, and 10% and kept therein for 60 minutes. Table 10 shows that the diameters of the beads decreased with increased conductivity of the CaCl₂ solution when the concentration was lower than 3%, and the hardness of the beads increased. After the aeration stress test, the beads that were hardened in solutions with concentrations between 0.5 and 2% maintained their white spherical appearance. Beads which were hardened in solutions with concentrations of 0.25%, 3% and higher became translucent after the aeration stress test. These gel beads have some leakage of microorganisms from the gel beads.

TABLE 10

| Lot No. | 151 | 152 | 153 | 154 | 155 | 156 | 157 |
|---|---|---|---|---|---|---|---|
| CaCl₂ | 0.25 | 0.5 | 1 | 2 | 3 | 5 | 10 |
| Concentration | | | | | | | |
| (%) | | | | | | | |
| Size | 3.87 ± | 3.57 ± | 3.27 ± | 3.11 ± | 2.98 ± | 3.28 ± | 3.45 ± |
| (mm) | 0.120 | 0.170 | 0.024 | 0.023 | 0.004 | 0.003 | 0.017 |
| Hardness | 0.082 ± | 0.198 ± | 0.235 ± | 0.289 ± | 0.302 ± | 0.287 ± | 0.315 ± |
| (kg/cm²) | 0.048 | 0.029 | 0.007 | 0.010 | 0.002 | 0.021 | 0.038 |
| Appearance* | Soft | White | White | White | Translucent | Translucent | Soft |
| | translucent | | | | | | translucent |

*Color of beads after stress test with coarse air bubble aeration

The properties of the gel beads of this example would be improved by application of the use of PU, etherification compounds and/or anions or anion releasing compounds to the making of the gel beads.

### Example 8

In this embodiment, a pilot plant was operated using this invention. A PVA-boric acid method was used, including the use of an aqueous solution (150 kg) containing 10% by weight of PVA that was mixed thoroughly with a concentrated sludge solution (3 kg) containing microorganisms (sludge concentration>6 g/L). The PVA gel beads were transferred into aqueous solutions of MgSO₄ with a conductivity of 155.3 mmho/cm and kept therein for 90 minutes. These beads were then removed from the solution and rinsed with water. The hardness of the beads was 0.44 kg/cm². The average diameter of the beads was 3.14±0.08 mm.

In this example, 150 kg of the beads were added into a 3.2-m³ airlift pilot bioreactor for advanced wastewater treatment in a petrochemical factory located in an Industrial Park. The wastewater to be treated was the effluent of the wastewater plant in the factory. The target effluent COD (chemical oxygen demand) concentration of the advanced treatment was below 250 mg/L to secure the effluent to meet the Industrial Management Center Wastewater Effluent Standard, COD below 480 mg/L. The hydraulic retention time was 20-24 hrs. The reaction was performed outdoors without temperature or pH control.

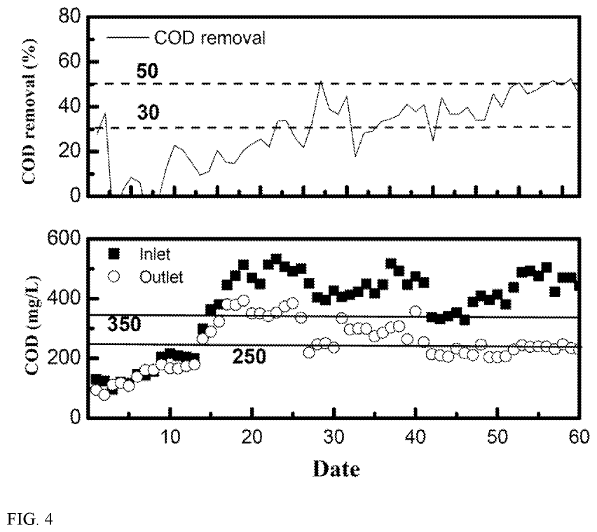

FIG. 4 shows that COD concentration in the wastewater was reduced to about 250 mg/L required by the factory during a 60-day operation and the removal efficiency eventually reached 50%. The COD removal efficiency is defined as the ratio of the amount of COD removed to the total COD originally in the wastewater. During the test, ten beads were removed from the system to measure their hardness every day. The hardness of the beads decreased from 0.43 kg/cm² to 0.23 kg/cm² after four days. However, the hardness of the beads increased to 0.41 kg/cm² on the eighth day, after which it remained between 0.40 to 0.70 kg/cm². The beads maintained their spherical shape and surface strength after the 60-day operation.

The field test failed once because the PVA gel beads dissolved. In the second trial a post treatment using MgSO₄ was adopted. These gel beads still have some leakage of PVA (less than 5%) from the gel beads that is not obvious in the wastewater. However, we could observe the foaming to see if there is leakage under the stress test. Without the sulfuric acid or PU step in the pretreatment, it was observed that foaming of about 12 cm occurred during the stress test. On the other hand, with gel beads with the sulfuric acid or PU pretreatment according to an embodiment of this invention, the foaming is less then 1 cm under a stress test. This indicates that with the pretreatment of embodiments of this invention, the leakage is minimized.

### Example 9

In another embodiment of a pilot plant, a PVA-boric acid method was used with immobilized sludge as in Example 8 except that the gel beads were transferred into a 1% of NaCl solution with a conductivity of 21.5 mmho/cm and kept therein for 120 minutes. The average diameter of the beads was 4.37±0.22 mm. About 15 kg of the beads were added into one of the dual 100-L bioreactors for a wastewater treatment test in a petrochemical factory. The same target wastewater for IS and SS was coming from the outlet of an anaerobic system of the factory's wastewater treatment system. The hydraulic retention time was 8 to 12 hours. The reaction was performed outdoors without temperature or pH control for three months.

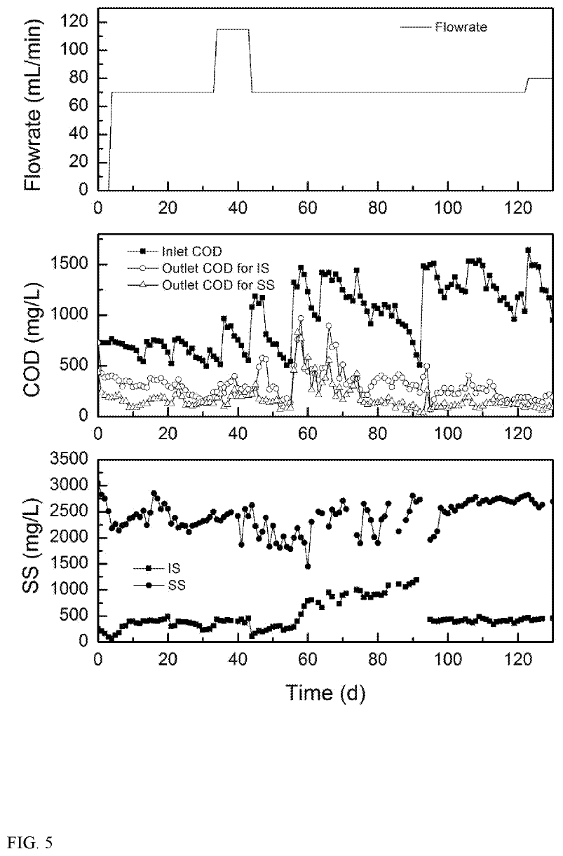

FIG. 5 shows that the immobilized gel beads could remove COD effectively even if the influent flowrate was increased to 120 mL/min (13.8 h retention time). The COD removal efficiency was defined as the ratio of the amount of COD removed to the total COD in the inlet of wastewater. The COD in FIG. 5 was measured without filtration of suspended solids in the beginning. It was corrected using 1-micron filter paper and the trend of COD from the two different systems was followed. For the suspension systems (SS), old activated sludge was added periodically to prevent washout of sludge. On the other side containing an embodiment of this invention, the suspended solid concentration in the outlet for the immobilized system (IS) was only ⅕ to ⅙ of the concentration in the SS. All the outlet suspended solids needed to be treated for recycle or disposal using a frame filter press with chemical added. Thus, the plant costs will be significantly higher for the chemical and electricity expenses for the SS. Furthermore, the less suspended solid production in IS will provide cost efficiencies in the disposal of the suspended solids.

FIG. 5 shows the difference of outlet COD between the IS and SS. IS always has higher COD than SS. It was originally hypothesized that the COD in IS was contributed by some leakage of microorganism from the gel beads. It could be observed by the naked eye. However, after reviewing the COD data that was pretreated by various size filtration before measurement, we found that only about 50 mg/L COD is contributed by the leakage of PVA. Although we can achieve the desired results to meet the effluent standard in this example, we still observe the leakage from the gel and gel beads which is not ideal. In some cases, this leakage results in a non-operable system. For example, in the biological laboratory setting using zebra fish to conduct genetic experiments, the water needs to be denitrified to keep the fish healthy. A leakage of these gel beads would be detrimental to the fish. A non-leakage of the gel and gel beads should not only clean up the water but also keep the fish alive.

Therefore, the properties of the gel beads discussed in this example for the plant treatment and the zebra fish would have been improved by the application of the PU, etherification and/or anions or anion releasing compounds of this invention.

### Example 10

In this embodiment, the unheated PU/PVA gel beads were used to cultivate algae, nitrifiers from a local petrochemical activated sludge system, and pure denitrifying culture purchased from Azoo (New Taipei City, Taiwan). The composition of PU/PVA gel beads is 10% PVA (36 g) and 2.3% PU (15g with 55% solid content) in the mixture of 285-mL reverse osmosis water and 60-mL microorganism solution (2 g/L). The results show the algal growth within 2-3 days. Cultivation of nitrifiers shows pink color in the bottom of the tank. The urea fed with 800 mg/L was utilized completely in a 1-liter air sparger during 3-day fed-batch cultivation. The denitrification process emitted nitrogen and the PU/PVA gel beads floated on the water surface. The concentration of NO₃ was completely utilized under 2-day fed-batch cultivation. This is evidence that the improvement of the physical and chemical structure of PU/PVA gel beads of this invention does not have the previous drawbacks and permits the mass transfer capability of immobilized substances such as microorganisms for being used in the biological field.

### LITERATURE

1. Dumitriu, S. and Chornet, E., Processes with immobilized enzymes and cells. In “Bioconversion of waste materials to industrial products.” Martin, A.M. (Ed.) New York: Springer, Pages 29-102, 1998.
2. Kuraray Co., Ltd., Shaped article made of porous hydrogel, method for producing the same and use thereof. TW201332640 A1, 2012.
3. Aslam, M., Kalyar, M.A., Raza, Z.A., Polyvinyl Alcohol: A Review of Research Status and Use of Polyvinyl Alcohol Based Nanocomposites. Polymer Engineering and Science. 58:2119-2132, 2018.
4. Hashimoto, S and Furukawa, K., Immobilization of activated sludge by PVA-boric acid method. Biotechnology & Bioengineering. 30(1):52-9, 1987.
5. Yao, L. and Swords, G.A., Hydrogel and methods of making and using same. U.S. Pat. No. 6,268,405 B1, 2001.
6. Ariga, O., Takagi, H., Nishizawa, H., Sano, Y., Immobilization of microorganisms with PVA hardened by iterative freezing and thawing. Journal of Fermentation Technology. 65:651-658, 1987.
7. Shinozaki, A. and Abe, K., Microorganism immobilized in a gel prepared from polyvinyl alcohol and a polysaccharide. U.S. Pat. No. 5,034,324, 1991.
8. Chen, K.-C. and Lin, Y.-F., Immobilization of microorganisms or enzymes in polyvinyl alcohol beads. U.S. Pat. No. 5,290,693, 1994.
9. Kitano, K., Production of microorganism-immobilized support. Japan Patent: JPS645491A, 1987.
10. Van Dinh, P. and Bach, L.T., Immobilized bacteria by using PVA (Polyvinyl alcohol) crosslinked with Sodium sulfate. The International Journal of Engineering Science., 7(1):41-47, 2014.
11. Schmidt, A., Balle, G., Eisfeld, K., Ether of polyvinyl alcohol. U.S. Pat. No. 1,971,662, 1934.
12. Prüße, U., Jahnz, U., Wittlich, P., Breford, J., Vorlop, K.-D., Bead production with JetCutting and rotating disk/nozzle technologies. Federal Research Center for Agriculture (FAL)- Brunswick (eds.), Völkenrode Agricultural Research, special issue 241, pp.1-11, 2002.
13. Huang, S.W., New method for producing immobilized microorganisms gel beads. U.S. provisional: 63/003,516, Apr. 1, 2020.
14. Hwang, S-.C., Lin, Y.H., Ho, Ho, H-.Y., Method for producing immobilized microorganisms gel beads. U.S. provisional: 62/856,328, Jun. 3, 2019.
15. Hwang, S-.C. J., Chang, H.M., Hsu, T.H., Treatment of the waste gas containing toluene and ethyl acetate by airlift bioreactor with PU/PVA immobilization cell beads. CHU-94-TR-05, 2005.
16. Hwang, S-.C. J., Lin, Y.H., Chen, P.A., Lee, M.C., Ho, H.Y., Wu, J.Y., Development of a novel microbial immobilization method using anionic polyurethane, R.O.C. Patent: 1425050, Feb. 1, 2014.
17. Wu PF, Teng JC, Lin YH, Hwang SCJ, Increasing algal biofuel production using Nannocholorpsis oculata cultivated with anaerobically and aerobically treated swine wastewater. Bioresource Technology. 133, p.102-108, 2013.
18. Chen KC, JY Wu, DJ Liou, SCJ Hwang, Decolorization of the textile dyes by newly isolated bacterial strains. Journal of Biotechnology. 101 (1), 57-68, 2003.
19. Chen KC, JY Wu, CC Huang, YM Liang, SCJ Hwang, Decolorization of azo dye using PVA-immobilized microorganisms. Journal of Biotechnology .101 (3), 241-252, 2003.
20. Chen KC, JY Wu, WB Yang, SCJ Hwang, Evaluation of effective diffusion coefficient and intrinsic kinetic parameters on azo dye biodegradation using PVA-immobilized cell beads. Biotechnology and Bioengineering. 83 (7), 821-832, 2003.
21. Wu JY, SCJ Hwang, CT Chen, KC Chen, Decolorization of azo dye in a FBR reactor using immobilized bacteria. Enzyme and Microbial Technology. 37 (1), 102-112, 2005.
22. Wu JY, KC Chen, CT Chen, SCJ Hwang, Hydrodynamic characteristics of immobilized cell beads in a liquid-solid fluidized-bed bioreactor. Biotechnology and Bioengineering. 83 (5), 583-594, 2003.
23. He SY, YH Lin, KY Hou, SCJ Hwang, Degradation of dimethyl-sulfoxide-containing wastewater using airlift bioreactor by polyvinyl-alcohol-immobilized cell beads. Bioresource Technology. 102 (10), 5609-5616, 2011.
24. Lin YH, SCJ Hwang, WC Shih, KC Chen, Development of a novel microorganism immobilization method using anionic polyurethane. Journal of Applied Polymer Science. 99 (3), 738-743, 2006.
25. Hwang SCJ, JY Wu, YH Lin, IC Wen, KY Hou, SY He, Optimal dimethyl sulfoxide biodegradation using activated sludge from a chemical plant. Process Biochemistry. 42 (10), 1398-1405, 2007.
26. Lin YH, SCJ Hwang, JY Wu, FY Chang, KC Chen, Simultaneous removal of carbon and nitrogen from swine wastewater using an immobilized-cell reactor. Journal of Environmental Engineering. 132 (3), 423-429, 2006.
27. Hwang SCJ, YH Lin, KS Huang, JY Lyuu, CT Hou, HH Chen, SY He, Treatment of acetone waste gases using slurry-phase airlift embedded with polyacrylamide-entrapped cell beads. Journal of the Air & Waste Management Association. 59 (10), 1230-1238, 2009.
28. Lin YH, HT Lee, HY Yin, SCJ Hwang, Method for producing butyric acid, butanol and butyrate ester. US Patent 9,371,548, 2012.
29. He SY, SCJ Hwang, YH Lin, KY Hou, Treatment of dimethyl-sulfoxide-containing optoelectronics wastewater using airlift bioreactor with PVA-immobilized cell beads. Chemical, Biological And Environmental Engineering. 156-160, 2010.

### Other Embodiments

Although the present invention has been described with reference to teaching, examples and preferred embodiments, one skilled in the art can easily ascertain its essential characteristics, and without departing from the spirit and scope thereof can make various changes and modifications of the invention to adapt it to various usages and conditions. Those skilled in the art will recognize or be able to ascertain using no more than routine experimentation, many equivalents to the specific embodiments of the invention described herein. Such equivalents are encompassed by the scope of the present invention.

All publications, patents, and applications mentioned in this specification are herein incorporated by reference.

What is claimed is:

1. A method for making gel or gel beads containing one or more immobilized substances comprising the following operations (a) through (e), done serially or combined together:
(a) forming a polyurethane (PU) and polyvinyl alcohol (PVA) slurry solution (PU/PVA slurry solution) containing one or more anions or anion releasing compounds;
(b) combining one or more substances to be immobilized with the PU/PVA slurry solution;
(c) combining a boric acid solution with the PU/PVA slurry solution with the one or more substances to be immobilized and forming PU/PVA gel or PU/PVA gel beads containing one or more immobilized substances;
(d) combining one or more hardening agents with the PU/PVA gel or PU/PVA gel beads containing the one or more immobilized substances; and
(e) optionally, combining one or more reinforcement agents with the PU/PVA gel or PU/PVA gel beads containing the one or more immobilized substances.

2. The method of claim 1, wherein the one or more anions or anion releasing compounds comprises sulfate, phosphate, and/or borate anions.

3. The method of claim 1, wherein forming the PU/PVA gel or PU/PVA gel beads in (c) can be performed by a dropping apparatus, an extruder or spreading on a surface.

4. The method of claim 1, wherein the one or more hardening agents comprises an alkali metal, an alkaline earth metal, other metal ion, and/or a mixture thereof.

5. The method of claim 4, wherein the alkali metal is selected from the group consisting of Li⁺, Na⁺, K⁺, and/or mixtures thereof.

6. The method of claim 4, wherein the alkaline metal is selected from the group consisting of Ca²⁺, Mg²⁺, and/or mixtures thereof.

7. The method of claim 4, wherein the other metal ion is selected from the group consisting of Al⁺³, Fe²⁺, Fe⁺³, Zn²⁺ and Cu²⁺, and/or mixtures thereof.

8. The method of claim 1, wherein the optional one or more reinforcement agents comprises (a) synthetic fibers selected from the group consisting of fibers of polyacrylic acid, polyvinyl acetate, polyacrylamide, and/or mixtures thereof, and/or (b) natural fibers selected from the group consisting of fibers from algae, cellulose, pulp, cotton, linen, and/or mixtures thereof.

9. The method of claim 1 wherein the substances to be immobilized are selected from the group consisting of microorganisms, cells, enzymes, non-enzyme chemicals, sludge, or mixtures of materials.

10. Gel or gel beads containing one or more immobilized substances made by the method of claim 1.

11. A method of purifying a substrate comprising:
(a) applying the gel or gel beads containing one or more immobilized substances of claim 10 to the substrate;
(b) purifying the substrate with the gel or gel beads containing one or more immobilized substances; and
(c) recovering the gel or gel beads containing one or more immobilized substances from the purified substrate.

12. A method of aqueous solution treatment, including wastewater treatment, aquaculture water treatment, aquarium water treatment, chemical process solution treatment and production, manufacturing process solution treatment and production, biofuel and biodiesel production, antibiotic process solution treatment or production, and/or other pharmaceutical process solution treatment or production, comprising:
(a) applying the gel or gel beads containing one or more immobilized substances of claim 10 to the aqueous solution;
(b) treating the aqueous solution by reducing chemical oxygen demand, reducing odor, denitrifying, nitrifying, and/or purifying the aqueous solution with the gel or gel beads containing one or more immobilized substances; and
(c) recovering the gel or gel beads containing one or more immobilized substances from the treated aqueous solution.

13. A method of gas treatment comprising:
(a) applying the gel or gel beads containing one or more immobilized substances of claim 10 to the gas;
(b) treating the gas by reducing volatile organic compounds, reducing odor, and/or purifying the gas with the gel or gel beads containing one or more immobilized substances; and
(c) recovering the gel or gel beads containing one or more immobilized substances from the treated gas.

14. A method of odor treatment of a substrate having an odor comprising:
(a) applying the gel or gel beads containing one or more immobilized substances of claim 10 to the substrate having an odor;
(b) treating the substrate having an odor by reducing the odor of the substrate having an odor with the gel or gel beads containing one or more immobilized substances; and
(c) recovering the gel or gel beads containing one or more immobilized substances from the treated substrate having an odor.

15. A method for making gel or gel beads containing one or more immobilized substances comprising the following operations (a) through (e), done serially or combined together:
(a) forming a polyvinyl alcohol (PVA) slurry solution (PVA slurry solution) containing one or more etherification compounds, and optionally containing one or more anions or anions releasing compounds;
(b) combining one or more substances to be immobilized with the PVA slurry solution;
(c) combining boric acid solution with the PVA slurry solution and forming PVA gel or PVA gel beads containing one or more immobilized substances;
(d) combining one or more hardening agents with the PVA gel or PVA gel beads containing one or more immobilized substances; and
(e) optionally, combining one or more reinforcement agents with the PVA gel or PVA gel beads containing one or more immobilized substances.

16. The method of claim 15, wherein the etherification compound in (a) is sulfuric acid or another acid.

17. The method of claim 15, wherein the one or more anions or anion releasing compounds comprises sulfate, phosphate, and/or borate anions.

18. The method of claim 15, wherein the pH of the PVA slurry solution in (a) is less than about pH 7.

19. The method of claim 15, wherein the pH of the PVA slurry solution in (a) is about pH 5.5.

20. The method of claim 15, wherein the pH of the PVA slurry solution in (a) is above about pH 3.

21. The method of claim 15, wherein forming PVA gel or PVA gel beads in (c) is performed with a dropping apparatus, an extruder or by spreading on a surface.

22. The method of claim 15, wherein the one or more hardening agents comprises an alkali metal, an alkaline earth metal, other metal ion, and/or a mixture thereof.

23. The method of claim 22, wherein the alkali metal is selected from the group consisting of Li⁺, Na⁺, K⁺, and/or mixtures thereof.

24. The method of claim 22, wherein the alkaline metal is selected from the group consisting of Ca²⁺, Mg²⁺, and/or mixtures thereof.

25. The method of claim 22, wherein the other metal ion is selected from the group consisting of Al⁺³, Fe²⁺, Fe⁺³, Zn²⁺ and Cu²⁺, at

[symbol]

26. The method of claim 15, wherein the optional one or more reinforcement agents comprises (a) synthetic fibers selected from the group consisting of fibers of polyacrylic acid, polyvinyl acetate, polyacrylamide, and/or mixtures thereof, and/or (b) natural fibers selected from the group consisting of fibers from algae, cellulose, pulp, cotton, linen, and/or mixtures thereof.

27. The method of claim 15 wherein the substances to be immobilized are selected from the group consisting of microorganisms, cells, enzymes, non-enzyme chemicals, sludge, or mixtures of materials.

28. Gel or gel beads containing one or more immobilized substances made by the method of claim 15.

29. A method of purifying a substrate comprising:
(a) applying the gel or gel beads containing one or more immobilized substances of claim 28 to the substrate;
(b) purifying the substrate with the gel or gel beads containing one or more immobilized substances; and
(c) recovering the gel or gel beads containing one or more immobilized substances from the purified substrate.

30. A method of aqueous solution treatment, including waste water treatment, aquaculture water treatment, aquarium water treatment, chemical process solution treatment and production, manufacturing process solution treatment and production, biofuel and biodiesel production, antibiotic process solution treatment or production, and/or other pharmaceutical process solution treatment or production, comprising:
(a) applying the gel or gel beads containing one or more immobilized substances of claim 28 to the aqueous solution;
(b) treating the aqueous solution by reducing chemical oxygen demand, reducing odor, denitrifying, nitrifying, and/or purifying the aqueous solution with the gel or gel beads containing one or more immobilized substances; and
(c) recovering the gel or gel beads containing one or more immobilized substances from the treated aqueous solution.

31. A method of gas treatment comprising:
(a) applying the gel or gel beads containing one or more immobilized substances of claim 28 to gas;
(b) treating the gas by reducing volatile organic compounds, reducing odor, and/or purifying the gas with the gel or gel beads containing one or more immobilized substances; and
(c) recovering the gel or gel beads containing one or more immobilized substances from the treated gas.

32. A method of odor treatment of a substrate having an odor comprising:
(a) applying the gel or gel beads containing one or more immobilized substances of claim 28 to a substrate having an odor;
(b) treating the substrate by reducing the odor of the substrate having an odor with the gel or gel beads containing one or more immobilized substances; and
(c) recovering the gel or gel beads containing one or more immobilized substances from the treated substrate having an odor.

33. A gel bead for use in an application comprising:
(a) linked polyvinyl alcohol (PVA) units;
(b) one or more immobilized substances, wherein the immobilized substances are selected from the group consisting of microorganisms, cells, enzymes and/or other materials;
(c) a size of about 3 mm to about 5 mm;
(d) a hardness of greater than or equal to about 0.03 kg/cm²; and
(e) wherein the gel bead has less than about 10% leakage of PVA or immobilized substances from the gel bead after one week of use of the gel bead in the application.

34. The gel bead of claim 33 wherein the gel bead also comprises linked polyurethane/PVA units.
